(19) United States
(12) Patent Application Publication
(10) Pub. No.: US 2014/0357563 A1
(43) Pub. Date: Dec. 4, 2014

(54) Compositions and Methods for the Generation of Activated Protein C and Methods of Use Thereof

(71) Applicant: The Children's Hospital of Philadelphia, Philadelphia, PA (US)

(72) Inventors: Mortimer Poncz, Narbeth, PA (US); Maria Anna Kowalska, Blue Bell, PA (US)

(21) Appl. No.: 14/363,989

(22) PCT Filed: Dec. 10, 2012
(86) PCT No.: PCT/US12/68743

§ 371 Date: Jun. 9, 2014

Related U.S. Application Data
(60) Provisional application No. 61/568,853, filed on Dec. 9, 2011.

(51) Int. Cl. A61K 38/36; A61K 31/727; A61K 45/06; A61K 38/16

(52) U.S. Cl. CPC A61K 38/366; A61K 38/16; A61K 38/36; A61K 31/727; A61K 45/06
USPC 514/13.8; 514/13.7; 514/56

(57) ABSTRACT

Compositions and methods for generating activated protein C and methods of use thereof are disclosed.

This application claims priority under 35 U.S.C. §119(e) to U.S. Provisional Patent Application No. 61/568,853, filed Dec. 9, 2011. The foregoing application is incorporated by reference herein.

This invention was made with government support under Grant Nos. 5P01HL040387-24 and 5R01HL084006-05 awarded by the National Institutes of Health. The Government has certain rights in this invention.

### FIELD OF THE INVENTION

The present invention relates to the field of hematology and related diseases and disorders. More specifically, the invention provides compositions and methods for the generation of activated protein C and methods of use thereof.

### BACKGROUND OF THE INVENTION

Several publications and patent documents are cited throughout the specification in order to describe the state of the art to which this invention pertains. Full citations of these references can be found throughout the specification. Each of these citations is incorporated herein by reference as though set forth in full.

Microbial infection leads to systemic activation of inflammatory and coagulation pathways that may result in sepsis with mortality rates as high as 60%. Indeed, sepsis, the systemic inflammatory response to infection, affects over 700,000 people/year in the United States costing billions in health care costs per year with nearly 250,000 deaths (Hotchkiss et al. (2003) N. Engl. J. Med., 348:138-50; Angus et al. (2001) Crit. Care Med., 29:1303-10). A number of clinical trials using naturally occurring anticoagulants have been tried including antithrombin and tissue factor (TF) pathway inhibitors. In patients with severe sepsis, generation of endogenous aPC is impaired, in part due to endothelial dysfunction with decreased levels of thrombomodulin (TM) and endothelial protein C receptor (EPCR). The clinical benefit of activated protein C (aPC) has also been demonstrated in the treatment of life-threatening sepsis. Despite recent advances in therapy, mortality remains high and new methods of treatment are needed.

### SUMMARY OF THE INVENTION

In accordance with the instant invention, methods of producing activated protein C (aPC) are provided. In a particular embodiment, methods for inhibiting, treating, preventing, and/or reducing a coagulopathy and/or the diseases or disorders associated therewith by increasing aPC in a subject are provided. In certain embodiments, the method comprises balancing the amount of negatively charged (anionic) compounds such as heparin and heparinoids with the amount of positively charged (cationic) compounds such as PF4 and histones to maximize the production of aPC. In a particular embodiment, the method comprises determining the concentration of PF4 and histones in a biological sample (e.g., blood or a fraction thereof) obtained from a subject, and administering to the subject a therapeutically effective amount of at least one negatively charged compound when the concentration of PF4 and histones is greater than the optimal or desired concentration for producing aPC or administering to the subject a therapeutically effective amount of at least one positively charged compound when the concentration of PF4 and histones is lower than the optimal or desired concentration for producing aPC. The methods may further comprise determining the concentration of heparin and/or heparinoids; aPC; and/or endothelial protein C receptor (EPCR). The methods may further comprise determining the optimal concentration of PF4 and/or other blood components in the biological sample for the formation of aPC. The methods may further comprise administering thrombomodulin with chondroitin sulfate side chains to the subject. In a particular embodiment, the coagulopathy is disseminated intravascular coagulopathy (DIC). In certain embodiments, the methods further comprise administering at least one therapeutic agent effective against the disorder or disease associated with the coagulopathy. In a particular embodiment, the methods further comprise the administration of at least one other anti-sepsis agent.

### BRIEF DESCRIPTION OF THE DRAWINGS

FIG. 1A provides a graph showing aPC activity at various PF4 concentrations. FIG. 1B provides a graph showing aPC activity (as fold change to the absence of PF4) at various heparin concentrations. Triangles represent PF4 at 12.5 μg/ml and diamonds represent PF4 at 400 μg/ml. FIG. 1C provides a graph showing aPC activity at various PRT concentrations. FIG. 1D provides a graph showing aPC activity (as fold change to the absence of PRT) at various heparin concentrations. Triangles represent PRT at 1 μg/ml and diamonds represent PRT at 20 μg/ml.

FIG. 2 shows that histones stimulate aPC generation in-solution by IIa/TM complex. aPC generation was monitored using the chromogenic substrate S2366. Reaction mixtures contained TM [FIG. 2A: TM_{CS} or FIG. 2B: TM_{-CS}] at either 0.5 nM (open symbols) or 40 nM (black symbols) at increasing concentration of mixed histones. Each curve shows the mean±1 standard deviation (SD) of for 4-5 experiments, each performed in duplicate. FIG. 2C provides a graph showing aPC activity at various histone or PF4 concentrations in the presence of thrombomodulin with a chondroitin sulfate side chain (TM_{+CS}). FIG. 2D provides a graph showing aPC activity at various histone or PF4 concentrations in the presence of thrombomodulin without a chondroitin sulfate side chain (TM_{-CS}). FIG. 2E provides a graph showing aPC activity at various histone concentrations. FIG. 2F provides a graph showing aPC activity at various heparin concentrations. Triangles represent histones at 3 μg/ml and diamonds represent histones at 75 μg/ml. For FIGS. 2E and 2F, n=3-5.

FIG. 3A provides a graph showing aPC activity at various histone concentrations, optionally, in the presence of PF4 (50 μg/ml). n=3-5. FIG. 3B provides a graph showing aPC activity at various histone concentrations in the presence of TM_{+CS} and PF4 (50 μg/ml). FIG. 3C provides a graph showing aPC activity at various heparin concentrations in the presence of TM_{+CS}, PF4 (50 μg/ml), and histones (75 μg/ml). FIG. 3D provides a graph showing aPC activity at various histone concentrations in the presence of TM_{-CS} and varying amounts of heparin. FIG. 3E provides a graph showing Gla-domainless aPC activity at various histone concentrations in the presence of TM_{+CS} and varying amounts of heparin.

FIG. 4 shows that histones stimulate aPC generation in vivo in mice following IIa injection. Mouse aPC measured in plasma 10 minutes after co-injection of IIa (80 U/kg) and increasing concentrations of histones in the absence (open circles) or presence of 5 mg/kg of PF4 (closed circles) in PF4^{KO} mice. N=3-4 per data point. Mean±1 SD are shown. *p<0.01 as compared to histone alone.

FIG. 5 shows the effect of PF4 on aPC generation in the presence of histones. aPC generation was monitored in the presence of various histones. Histones were added in the absence (open symbols) or presence (grey symbols) of PF4 (25 μg/ml). Circles denote addition of mixed histones. Squares represent pure histone H1 (M.W. 20.7 kD) and diamonds histone H4 (M.W. 11.2 kD). N=3, each done in duplicate. Mean±1 SD shown.

FIG. 6 shows that PF4 and heparin modulate histone-dependent effects on PC activation. aPC generation was monitored in the presence of 0.5 nM of TM_{CS}. FIG. 6A: Histones were added in the absence (open circle) or presence of PF4 at low (3 μg/ml, grey diamonds) or peak (25 μg/ml, black squares) concentrations. FIG. 6B: Histones were added in the absence (open circle) or presence (black circles) of heparin (5 μg/ml). FIG. 6C: Same as in FIG. 6B but in the presence of 50 μg/ml of PF4 (no heparin, gray squares), (heparin 5 μg/ml, black squares). N=3, each done in duplicate. Mean±1 SD shown.

FIG. 7 shows that heparinoids affect aPC generation differently in the presence of low and high concentrations of histones. Increasing concentration of histones were added to the assay mix containing control buffer (circles) or heparinoids: 2 μg/ml (diamonds), 5 μg/ml (squares) or 10 μg/ml (triangles). FIG. 7A: UFH (open symbols) or FIG. 7B: ODSH (closed symbols). N=3, each done in duplicate. Mean±1 SD shown.

FIG. 8 shows that histones stimulate of aPC generation in vivo in mouse plasma—effect of UFH and ODSH. Mice were injected with buffer or 50 mg/kg of UFH or ODSH followed by histones 10 minutes later as indicated. 30 minutes later blood was collected. Mouse aPC was measured in plasma. Mean±1 SD are shown.

FIG. 9 shows the effect of UFH or ODSH on aPTT in the absence or presence of histones. Blood was collected from cut tails 20 minutes after injection of heparinoids and aPTT measured in plasma. FIG. 9A: WT mice were injected with control buffer (at 0 mg/kg heparanoid concentrations) or UFH (dark squares) or ODSH (open triangles) at the concentrations indicated. FIG. 9B: WT mice were injected with control buffer (open circles) or 50 mg/kg of either UFH (black square) or ODSH (gray triangles) 10 minutes before injection of increasing concentrations of histones. N=5 mice per data point. Mean±1 SD are shown.

### DETAILED DESCRIPTION OF THE INVENTION

Activated protein C (aPC) reduces the risk of death in disseminated intravascular coagulopathy (DIC) as seen in sepsis (Bernard et al. (2001) N. Engl. J. Med., 344:699-709; Bernard et al. (2004) Chest 125:2206-2216), while released histones increase that risk (Xu et al. (2009) Nat. Med., 15:1318-1321), and heparin may also influence the outcome (Kienast et al. (2006) J. Thromb. Haemost., 4:90-97; Levi et al. (2007) Am. J. Respir. Crit. Care Med., 176:483-490). Thrombomodulin (TM) in complex with thrombin (IIa) activates Protein C to aPC. TM is an anionic molecule due to posttranslational O-linked glycanation by and without CS, although the CS is labile.

Herein, it was observed that platelet factor 4 (PF4), which is released from platelets in large amounts in sepsis, regulates aPC generation following a bell-shaped curve in vitro: it increases aPC generation by TM_{+CS}/IIa complex at low concentration and inhibits it at high concentration. This activity by PF4 depends on its complexing with TM's CS. There is, therefore, an optimal PF4 concentration for maximum aPC, if TM_{+CS} is present. Heparin and heparinoids binds avidly to PF4, and depending on the ambient concentration of PF4, either inhibits or potentiates PF4 effect on aPC generation essentially moving the effective PF4 concentration along the same bell-shaped curve. Thus, heparin and/or other heparinoids can increase aPC generation (when the PF4 concentration is above peak concentration) and decrease aPC generation (when the PF4 concentration is below peak concentration).

Besides PF4, it is shown herein that histones have the same bell-shaped curve effect on aPC generation. Further, it is shown that the sum total of PF4 and histones have an additive effect on aPC generation. Accordingly, if one has high levels of PF4 already released, extra histones would only decrease aPC generation by TM_{+CS}/IIa complexes. It is also demonstrated herein that heparin and/or heparinoids affect the generation of aPC in the presence of histones or histones with PF4 in a similar fashion to PF4 alone. Specifically, heparin and/or heparinoids increases aPC generation when the sum of histone plus PF4 begins above the peak total concentration and decreases aPC generation if the sum total is below the peak.

Based on these findings, it is evident that monitoring PF4 and histone effective levels is beneficial in patients with disseminated intravascular coagulopathy as seen in sepsis or severe local inflammation/coagulopathy such as in ARDS (acute respiratory distress syndrome) to improve patient survival. In certain embodiments, the levels of heparin and/or other heparin-like molecules (e.g., heparinoids); aPC; and/or endothelial protein C receptors (EPCR) are also measured to determine the effective levels of PF4 and histones. This is in contrast to Ammollo et al. (J. Thromb. Haemost. (2011) 9:1795-803) which indicates that the GAG moiety of TM is not of clinical relevance and that histones have very different effects on TM-protein C functions than PF4.

As stated hereinabove, platelet factor 4 (PF4) increases activated protein C (aPC) generation through the thrombin (IIa)/thrombomodulin (TM) complex, both in vitro and in vivo. It is demonstrated herein that potentiation of aPC generation by PF4 requires the TM-glycosaminoglycan (GAG) domain. PF4 released from platelets in mice enhances aPC generation in a model of IIa infusion and released PF4 can protect against lipopolysaccharide-induced endotoxemia. Endotoxin, the lipopolysaccharide (LPS) associated with the membranes of gram-negative bacteria, activates monocytes. Activated monocytes express tissue factor (TF) that initiates thrombin (IIa) generation. Generation of aPC by IIa is accelerated in vitro and in vivo by its binding to TM on the surface of endothelial cells. TM is an anionic molecule due to posttranslational addition of variable amounts of O-linked chondroitin sulfate (CS) GAG. It exists in two major glycoforms, one that contains CS adduct and one that is CS free, which differ in specific activity.

By increasing aPC generation, PF4 can impact outcome in sepsis. Herein, it is demonstrated that the effect of PF4 on aPC generation follows a biphasic curve when tested in solution on human TM expressing HEK293 and on primary endothelial cells (ECs) with a peak concentration at around 25 μg/ml. Formation of complexes at a specific molar ratio between positively charged tetramers of PF4 and negatively charged chondroitin sulfate (CS) on the TM glycosaminoglycan (GAG) is important for the increase in aPC generation. Other positively-charged molecules like protamine sulfate (PRT) affect aPC generation in a similar manner, and heparin, which is known to bind PF4 more avidly than CS, lowers effective PF4 or PRT concentrations. Herein, it is also demonstrated that histones, which are small positively-charged molecules, affect aPC generation. Notably, histones are released from cells in sepsis. Histones are cytotoxic toward endothelial cells and are lethal when injected into mice. However, aPC reverses this lethality. In vitro experiments were performed both in solution and with TM-expressing cells, in the absence or presence of endothelial protein C receptor. It was determined that individual or mixed histones affect aPC formation following a similar biphasic curve seen with PF4, with a peak effect at around 10 μg/ml. Notably, the effect of histones was to a lower extent than PF4 (approximately 2 times maximal increase compared to 6 times for PF4). Additionally, it was also determined that histones and PF4 acted additively at low concentrations. Significantly, histones decreased aPC generation when tested in the presence of optimal PF4 concentration (25 μg/ml). Just as with PF4, added heparin and/or heparinoids effectively decreased histone concentration and shifted the curve for aPC generation to the right, both in the absence or presence of PF4. Accordingly, PF4 released from platelets normally augments aPC generation and low concentrations of histones have a similar effect. However, when histones are released in sepsis in high concentrations, their interaction with CS on thrombomodulin blocks formation of complexes between PF4 and TM's CS that are optimal for maximal increase of aPC generation.

In addition to the above, the effect of histones on aPC generation was tested in vivo. Injection of histones in mice increased IIa-induced (2 U/kg) aPC generation in plasma. This increase was concentration dependent (at 1 to 20 mg/kg increasing aPC generation up to 10 times), but injection of higher amount of histones (40 mg/kg) became lethal. Mice that were overexpressing human PF4 had an increased lethality when histones at 40 mg/kg were co-injected with thrombin (2 U/kg) over the littermate mice deficient in murine PF4 (60% vs. 0% mortality, respectively, n=5 for each group) indicating that in vivo histones also act additively with PF4 on aPC generation. Accordingly, in severe septic patients, especially those with high platelet PF4 content, released histones suppress aPC generation because of the concurrent presence of excess free PF4 from activated platelets. By binding to the excess of PF4 and/or histones, administration of negatively charged molecules such as heparin and/or heparinoids will be beneficial in severe sepsis by allowing improved aPC generation. Indeed, in order to achieve optimal generation of activated Protein C, the balance of positively and negatively charged proteins is needed. This balance can be substantially changed when histones are released and platelets are activated releasing additional PF4 (e.g., during sepsis).

In accordance with the instant invention, methods of treating, preventing (e.g., inhibiting the onset), and/or reducing the severity of coagulopathy in a subject in need thereof are provided. The coagulopathy may be disseminated or localized. Coagulopatic related diseases or disorders that may be treatable by the methods of the instant invention include, without limitation: vascular diseases and inflammatory responses, thrombosis, deep venous thrombosis, arterial thrombosis, post surgical thrombosis, tissue ischemia, ischemic peripheral vascular disease, hyperoxic injury, complications or disorders associated with application of a coronary artery bypass graft (CABG), complications or disorders associated with percutaneous transdermal coronary angioplastry (PTCA), stroke, tumor metastasis, inflammation, septic shock, hypotension, acute lung injury (ALI), acute respiratory distress syndrome (ARDS), pulmonary embolism, disseminated intravascular coagulation (DIC), sepsis, systemic inflammatory response syndrome (SIRS), vascular restenosis, platelet deposition, myocardial infarction, acute myocardial infarction (AMI), and angiogenesis-related disorders. In a particular embodiment, the disease or disorder is systemic inflammatory response syndrome, acute lung injury, acute respiratory distress syndrome, disseminated intravascular coagulation, sepsis, and/or multiple organ failure in association with any of the preceding syndromes. In a particular embodiment, the coagulopathy is disseminated intravascular coagulopathy.

Disseminated intravascular coagulopathy (DIC) is also referred to as consumptive coagulopathy and disseminated intravascular coagulation. DIC is a complex systemic thrombohemorrhagic disorder involving the generation of intravascular fibrin and the consumption of procoagulants and platelets. The resultant clinical condition is characterized by intravascular coagulation and hemorrhage. DIC may be defined as an acquired syndrome characterized by the intravascular activation of coagulation with loss of localization arising from different causes and generally involving activation of systemic inflammation. DIC is most commonly observed in severe sepsis and septic shock and its severity correlates with mortality in severe sepsis.

Although bacteremia, including both gram-positive and gram-negative organisms, is most commonly associated with DIC, other organisms including viruses, fungi, and parasites may cause DIC. Trauma, especially neurotrauma, is also frequently associated with DIC, particularly in those patients with trauma who develop the systemic inflammatory response syndrome. Conditions which cause DIC include, without limitation: trauma (e.g., severe trauma, neurological trauma (e.g., acute head injury)), sepsis, tumor metastasis, malignancy, cancer, cancer treatment by chemotherapy or radiation therapy, transplant rejection, transfusion reaction, obstetric complication, vascular aneurysm, hepatic failure (e.g., liver disease), heat stroke, burn, shock, radiation exposure, infection (bacterial, viral, fungal, parasitic), and severe toxic reaction (e.g., snake bite, insect bite, transfusion reaction).

In a particular embodiment, methods of increasing aPC generation in a patient are provided (e.g., for the treatment, prevention, inhibition and/or reduction of a coagulopathy in a subject). In a certain embodiments, the method comprises measuring the concentration of PF4 in a biological sample obtained from a subject (e.g., animal or human), wherein a level of PF4 concentration greater than the optimal concentration for producing aPC (e.g., the concentration at which the most aPC is produced) indicates that the aPC can be increased in the subject by administering at least one negatively charged compound or protein such as TM_{CS} or heparin and/or heparinoids (e.g., variants of heparin and/or heparinoid that do not influence coagulation (e.g., de-N-sulfated heparin sodium salt or oxygen desulfated heparin (ODSH; e.g., 2-O,3-O desulfated heparin))) and wherein a level of PF4 concentration lower than the optimal concentration for producing aPC indicates that the aPC can be increased in the subject by administering at least one positively charged compound or protein such as PT, PF4 and/or histones (single or mixed), particularly PF4. In a particular embodiment, the method further comprises determining the optimal concentration of PF4 and/or other blood components for the production of aPC. The concentration of PF4 (and/or other components) for maximal aPC production can be determined by varying the amount of PF4 and/or other blood components (e.g., heparin/heparinoid, histones, EPCR, etc.) in human blood or a fraction thereof (optionally obtained from the subject to be treated) and measuring aPC formation (e.g., as described hereinblow). In a particular embodiment, at least one negatively charged compound or protein is delivered when the concentration of PF4 is at a concentration equal to or greater than the high concentration of PF4 observed in patients with severe sepsis. In a particular embodiment, at least one positively charged compound or protein is delivered when the concentration of PF4 is at a concentration equal to or lower than the concentration of PF4 observed in normal (healthy) subjects. In a particular embodiment, the concentration of PF4 is 12±5 ng/10⁶ platelets, 0.36±0.25 ng/μl of plasma, or 7-30, particularly 7-25 or 10-20, IU/10⁸ platelets (e.g., Peterson et al. (2010) Am. J. Hematol., 85:487-493; Lambert et al. (2007) Blood 110:1153-1160). The methods may further comprise the administration of TM_{+CS} to the subject. The above methods may further comprise measuring the amount of heparin and/or heparinoids, EPCR, and/or aPC present in the biological sample and modulating the administered compound (e.g., heparin/heparinoid) accordingly. In a particular embodiment, the biological sample is blood or a fraction thereof (e.g., plasma or serum).

In certain embodiments, the method comprises measuring the concentration of PF4 and histones (single or mixed) in a biological sample obtained from a subject (e.g., animal or human), wherein a level of PF4 and histone concentrations greater than the optimal concentration for generating aPC indicates that the aPC can be increased in the subject by administering at least one negatively charged compound or protein such as TM_{CS} or heparin and/or heparinoids (e.g., variants of heparin and/or heparinoid that do not influence coagulation (e.g., de-N-sulfated heparin sodium salt or oxygen desulfated heparin (ODSH; e.g., 2-O,3-O desulfated heparin))) and wherein a level of PF4 and histones concentration lower than the optimal concentration for producing aPC indicates that the aPC can be increased in the subject by administering at least one positively charged compound or protein such as PRT, PF4 and/or histones (single or mixed), particularly PF4. In a particular embodiment, the method further comprises determining the optimal concentration of PF4 and/or other blood components (e.g., histones) for the production of aPC. The concentration of PF4 (and/or other components) for maximal aPC production can be determined by varying the amount of PF4 and/or other blood components (e.g., heparin/heparinoid, histones, EPCR, etc.) in human blood or a fraction thereof (optionally obtained from the subject to be treated) and measuring aPC formation (e.g., as described hereinblow). In a particular embodiment, at least one negatively charged compound or protein is delivered when the concentration of PF4 and histones is at a concentration equal to or greater than the high concentration of PF4 and histones observed in patients with severe sepsis. In a particular embodiment, at least one positively charged compound or protein is delivered when the concentration of PF4 and histones is at a concentration equal to or lower than the concentration of PF4 and histones observed in normal (healthy) subjects. In a particular embodiment, the concentration of PF4 is 12±5 ng/10⁶ platelets, 0.36±0.25 ng/μl of plasma, or 7-30, particularly 7-25 or 10-20, IU/10⁸ platelets (e.g., Peterson et al. (2010) Am. J. Hematol., 85:487-493; Lambert et al. (2007) Blood 110:1153-1160). The methods may further comprise the administration of TM_{+CS} to the subject. The above methods may further comprise measuring the amount of heparin and/or heparinoids, EPCR, and/or aPC present in the biological sample and modulating the administered compound (e.g., heparin/heparinoid) accordingly. In a particular embodiment, the biological sample is blood or a fraction thereof (e.g., plasma or serum).

In accordance with the instant invention, methods of treating, preventing, and/or reducing the severity of coagulopathy in a subject in need thereof are provided. The coagulopathy may be disseminated or localized. In a particular embodiment, the coagulopathy is sepsis or ARDS. In a certain embodiments, the method comprises measuring the concentration of PF4 in a biological sample obtained from a subject (e.g., animal or human) and administering to the subject a therapeutically effective amount of at least one negatively charged compound or protein such as TM_{CS} or heparin and/or heparinoids (e.g., variants of heparin and/or heparinoid that do not influence coagulation (e.g., de-N-sulfated heparin sodium salt or oxygen desulfated heparin (ODSH; e.g., 2-O,3-O desulfated heparin))) when the concentration of PF4 was determined to be greater than the optimal concentration for producing aPC or administering a therapeutically effective amount of at least one positively charged compound or protein such as PRT, PF4 and/or histones (single or mixed), particularly PF4, when the concentration of PF4 was determined to be lower than the optimal concentration for producing aPC. In a particular embodiment, the method further comprises determining the optimal concentration of PF4 and/or other blood components for the production of aPC. The concentration of PF4 (and/or other components) for maximal aPC production can be determined by varying the amount of PF4 and/or other blood components (e.g., heparin/heparinoid, histones, EPCR, etc.) in human blood or a fraction thereof (optionally obtained from the subject to be treated) and measuring aPC formation (e.g., as described hereinblow). In a particular embodiment, at least one negatively charged compound or protein is delivered when the concentration of PF4 is at a concentration equal to or greater than the high concentration of PF4 observed in patients with severe sepsis. In a particular embodiment, at least one positively charged compound or protein is delivered when the concentration of PF4 is at a concentration equal to or lower than the concentration of PF4 observed in normal (healthy) subjects. In a particular embodiment, the concentration of PF4 is 12±5 ng/10⁶ platelets, 0.36±0.25 ng/μl of plasma, or 7-30, particularly 7-25 or 10-20, IU/10⁸ platelets (e.g., Peterson et al. (2010) Am. J. Hematol., 85:487-493; Lambert et al. (2007) Blood 110:1153-1160). The methods may further comprise the administration of TM_{+CS} to the subject. The above methods may further comprise measuring the amount of heparin and/or heparinoids, EPCR, and/or aPC present in the biological sample and modulating the administered compound (e.g., heparin/heparinoid) accordingly. In a particular embodiment, the biological sample is blood or a fraction thereof (e.g., plasma or serum).

In certain embodiments, the method comprises measuring the concentration of PF4 and histones (single or mixed) in a biological sample obtained from a subject (e.g., animal or human), wherein a concentration of PF4 and histone greater than the optimal concentration for generating aPC indicates that the aPC can be increased in the subject by administering at least one negatively charged compound or protein such as TM_{CS} or heparin and/or heparinoids (e.g., variants of heparin and/or heparinoid that do not influence coagulation (e.g., de-N-sulfated heparin sodium salt or oxygen desulfated heparin (ODSH; e.g., 2-O,3-O desulfated heparin))) and wherein a concentration of PF4 and histones lower than the optimal concentration for producing aPC indicates that the aPC can be increased in the subject by administering at least one positively charged compound or protein such as PRT, PF4 and/or histones (single or mixed), particularly PF4. In a particular embodiment, the method further comprises determining the optimal concentration of PF4 and/or other blood components for the production of aPC. The concentration of PF4 (and/or other components) for maximal aPC production can be determined by varying the amount of PF4 and/or other blood components (e.g., heparin/heparinoid, histones, EPCR, etc.) in human blood or a fraction thereof (optionally obtained from the subject to be treated) and measuring aPC formation (e.g., as described hereinblow). In a particular embodiment, at least one negatively charged compound or protein is delivered when the concentration of PF4 and histones is at a concentration equal to or greater than the high concentration of PF4 and histones observed in patients with severe sepsis. In a particular embodiment, at least one positively charged compound or protein is delivered when the concentration of PF4 and histones is at a concentration equal to or lower than the concentration of PF4 and histones observed in normal (healthy) subjects. In a particular embodiment, the concentration of PF4 is 12±5 ng/10⁶ platelets, 0.36±0.25 ng/μl of plasma, or 7-30, particularly 7-25 or 10-20, IU/10⁸ platelets (e.g., Peterson et al. (2010) Am. J. Hematol., 85:487-493; Lambert et al. (2007) Blood 110:1153-1160). The methods may further comprise the administration of TM_{+CS} to the subject. The above methods may further comprise measuring the amount of heparin and/or heparinoids, EPCR, and/or aPC present in the biological sample and modulating the administered compound (e.g., heparin/heparinoid) accordingly. In a particular embodiment, the biological sample is blood or a fraction thereof (e.g., plasma or serum).

With regard to the above methods, the concentration of the above markers can be determined by any method known in the art. The concentrations can be determined directly (e.g., direct binding with an antibody) or indirectly (e.g., an assay measuring activity). For example, the concentration of the above markers may be measured by immunoassay such as by ELISA. With regard to heparin and/or heparinoids, the amount of heparin and/or heparinoids may be measured indirectly, such as with activated partial thromboplastin time (aPTT) and anti-factor Xa activity assays.

Additionally, the above methods may further comprise assaying the biological sample in an aPC assay (e.g., those described in the examples below) with varying amounts of the agents in order to determine the optimal concentration for aPC generation.

In accordance with the instant invention, methods of treating, preventing, and/or reducing the severity of coagulopathy in a subject in need thereof are provided comprising the administration of TM_{+CS} to the subject. The coagulopathy may be disseminated or localized. In a particular embodiment, the coagulopathy is sepsis or ARDS. In a particular embodiment, the biological sample is blood or a fraction thereof (e.g., plasma or serum). In certain embodiment, the TM_{+CS} administered to the subject is in a composition which lacks or substantially lacks TM-CS. For example, the TM administered to the subject may be at least 75%, 80%, 85%, 90%, 95%, 97%, 99%, or 100% TM_{+CS}. In certain embodiment, additional therapeutic agents (e.g., anti-sepsis agents) may be co-administered with the TM_{+CS}.

In accordance with the instant invention, therapeutic agents (e.g., proteins) are administered to a patient to inhibit, treat, and/or prevent coagulopathy and the diseases or disorders associated with the coagulopathy. While delivery of the proteins directly to the subject is exemplified, the instant invention also encompasses the administration of nucleic acid molecules encoding the proteins (e.g., gene therapy) or cells expressing the proteins.

The therapeutic agents of the instant invention may be prepared in a variety of ways, according to known methods. For example, the therapeutic agents may be purified from appropriate sources, e.g., transformed bacteria, cultured animal cells or tissues, or animals (e.g., by immunoaffinity purification methods). The therapeutic agents may also be chemically synthesized.

The availability of nucleic acid molecules encoding the proteins enables production of the protein using in vitro expression methods and cell-free expression systems known in the art. Alternatively, larger quantities of the proteins may be produced by expression in a suitable prokaryotic or eukaryotic system. For example, a DNA molecule encoding for the proteins may be inserted into a plasmid vector adapted for expression in a bacterial cell, such as E. coli. Such vectors comprise the regulatory elements necessary for expression of the DNA in the host cell positioned in such a manner as to permit expression of the DNA in the host cell. Such regulatory elements required for expression include promoter sequences, transcription initiation sequences and, optionally, enhancer sequences. The proteins produced by gene expression in a recombinant prokaryotic or eukaryotic system may be purified according to methods known in the art. A commercially available expression/secretion system can be used, whereby the recombinant protein is expressed and secreted from the host cell, and readily purified from the surrounding medium by any method known in the art. For example, the recombinant protein may be purified by affinity separation, such as by immunological interaction with antibodies that bind specifically to the recombinant protein or nickel columns for isolation of recombinant proteins tagged with 6-8 histidine residues at their N-terminus or C-terminus. Alternative tags may comprise the FLAG epitope or the hemagglutinin epitope. Proteins prepared by the aforementioned methods may be analyzed according to standard procedures. For example, such protein may be subjected to amino acid sequence analysis, according to known methods.

The therapeutic agents of the instant invention will generally be administered to a patient (i.e., human or animal subject) in a composition with a pharmaceutically acceptable carrier. For example, therapeutic agents may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of therapeutic agents in the chosen medium may be varied and the medium may be chosen based on the desired route of administration of the pharmaceutical preparation. Except insofar as any conventional media or agent is incompatible with the therapeutic agents, its use in the pharmaceutical preparation is contemplated.

In yet another embodiment, the pharmaceutical compositions of the present invention can be delivered in a controlled release system, such as using an intravenous infusion, an implantable osmotic pump (e.g., a subcutaneous pump), a transdermal patch, liposomes, or other modes of administration. In another embodiment, polymeric materials may be employed. In yet another embodiment, a controlled release system can be placed in proximity of the target tissues of the animal, thus requiring only a fraction of the systemic dose. In particular, a controlled release device can be introduced into an animal in proximity to the desired site.

Selection of a suitable pharmaceutical preparation will also depend upon the mode of administration chosen. For example, the therapeutic agents may be administered by direct injection into an area proximal to the infection or may be delivered systemically. When delivered by direct injection, a pharmaceutical preparation comprises the therapeutic agents dispersed in a medium that is compatible with the site of injection. The therapeutic agents may be administered by any method such as intravenous injection into the blood stream, oral administration, or by subcutaneous, intramuscular or intraperitoneal injection. Pharmaceutical preparations for injection are known in the art. If injection is selected as a method for administering the therapeutic agents, steps should be taken to ensure that sufficient amounts of the molecules reach their target cells to exert a biological effect.

Pharmaceutical compositions containing the therapeutic agents of the instant invention as the active ingredient in intimate admixture with a pharmaceutically acceptable carrier can be prepared according to conventional pharmaceutical compounding techniques. The carrier may take a wide variety of forms depending on the form of preparation desired for administration, e.g., intravenous, direct injection, and intraperitoneal.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

The pharmaceutical preparation comprising the active ingredient may be administered at appropriate intervals, for example, at least twice a day or more until the pathological symptoms are reduced or alleviated, after which the dosage may be reduced to a maintenance level. The appropriate interval in a particular case would normally depend on the condition of the patient.

Pharmaceutical compositions comprising a therapeutic agent of the instant invention may be administered in coordination with at least one other agent used to treat the coagulopathy or sepsis. For example, other agents used to treat sepsis include, without limitation, anti-thrombin III, activated protein C (e.g., XIGRIS® (drotrecogin alfa); particularly for severe sepsis), BPI, anti-infectives, antibiotics (including, without limitation, β-lactams (e.g., penicillins and cephalosporins), vancomycins, bacitracins, macrolides (e.g., erythromycins), lincosamides (e.g., clindomycin), chloramphenicols, tetracyclines (e.g., immunocycline, chlortetracycline, oxytetracycline, demeclocycline, methacycline, doxycycline and minocycline), aminoglycosides (e.g., gentamicin, amikacins, and neomycins), amphotericins, cefazolins, clindamycins, mupirocins, sulfonamides and trimethoprim, rifampicins, metronidazoles, quinolones, novobiocins, polymixins, gramicidins, vancomycin, imipenem, meropenem, cefoperazone, cefepime, penicillin, nafcillin, linezolid, aztreonam, piperacilliri, tazobactam, ampicillin, sulbactam, clindamycin, metronidazole, levofloxacin, a carbapenem, linezolid, rifampin, and metronidazole), and agents which alleviate/treat the symptoms associated with sepsis (supportive care; e.g., cardiovascular support, respiratory support, renal replacement therapy, glucose control, and analgesia (see, e.g., www.sepsis.com). The therapeutic agents of the instant invention and the other anti-sepsis agents may be administered together in a single composition or may be administered in separate compositions. Additionally, the therapeutic agents of the instant invention and the other anti-sepsis agents may be administered at the same time or on different schedules.

### DEFINITIONS

The singular forms “a,” “an,” and “the” include plural referents unless the context clearly dictates otherwise.

The term “sepsis” as used herein refers to the systemic inflammatory response to infection. In other words, “sepsis” may refer to a systemic inflammatory response plus a documented infection (e.g., a subsequent laboratory confirmation of a clinically significant infection such as a positive culture for an organism) (see, e.g., American College of Chest Physicians Society of Critical Care Medicine (1997) Chest 101:1644-1655). The “systemic inflammatory response” is the body's overwhelming response to a noxious stimulus. The current definition is characterized by the following non-specific changes in the adult human body: 1) fast heart rate (tachycardia, heart rate>90 beats per minute), 2) low blood pressure (systolic<90 mmHg or MAP<65 mmHg), 3) low or high body temperature (<36 or >38° C.), 4) high respiratory rate (>20 breaths per minute), and 5) low or high white blood cell count (<4 or >12 billion cells/liter). When an identified infectious pathogen causes the inflammatory response, the resultant inflamed state is referred to as sepsis. Infectious agents which can cause sepsis include bacteria, viruses, fungi, and parasites.

As used herein, the term “sepsis” includes all stages of sepsis including, but not limited to, the onset of sepsis, severe sepsis, septic shock and multiple organ dysfunction associated with the end stages of sepsis. The “onset of sepsis” refers to an early stage of sepsis (e.g., prior to a stage when conventional clinical manifestations are sufficient to support a clinical suspicion of sepsis). “Severe sepsis” refers to sepsis associated with organ dysfunction (Bota et al. (2002) Intens. Care Med. 28:1619-1624; Ferreira et al. (2005) J. Amer. Med. Assoc. 286:1754-1758), hypoperfusion abnormalities, or sepsis-induced hypotension. Hypoperfusion abnormalities include, but are not limited to, lactic acidosis, oliguria, or an acute alteration in mental status. “Septic shock” refers to sepsis-induced hypotension that is not responsive to adequate intravenous fluid challenge and with manifestations of peripheral hypoperfusion. “Septic shock” may also be defined as severe sepsis accompanied by acute circulatory failure characterized by persistent arterial hypotension (a systolic arterial pressure below 90 mm Hg).

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition (e.g., a coagulopathy or sepsis) resulting in a decrease in the probability that the subject will develop the condition.

The phrase “effective amount” refers to that amount of therapeutic agent that results in an improvement in the patient's condition.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), water, aqueous solutions, oils, bulking substance (e.g., lactose, mannitol), excipient, auxilliary agent or vehicle with which an active agent of the present invention is administered. Suitable pharmaceutical carriers are described in “Remington's Pharmaceutical Sciences” by E.W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., IIandbook of Pharmaceutical Excipients, American Pharmaceutical Association, Washington.

The following examples are provided to illustrate various embodiments of the present invention. The examples are illustrative and are not intended to limit the invention in any way.

### Example 1

### Materials and Methods

### In Vitro aPC Assay

Generation of aPC was assayed in 96-well plates as described previously (Slungaard et al. (1994) J. Biol. Chem., 269:25549-25556; Slungaard et al. (1993) J. Clin. Invest., 91:1721-1730). Briefly, thrombomodulin (TM) was mixed with various amounts of PF4 and/or heparin for 10 minutes in assay buffer (final concentration, 20 mM Tris, 100 mM NaCl, 1 mM CaCl₂, 0.1% BSA, pH 7.5). Protein C (PC) was added for an additional 10 minutes followed by addition of thrombin (IIa) at a final concentration of either 0.2 or 2 nM and 15 minutes of incubation. All incubations were done at 37° C. Final concentrations of TM and PC were 0.5 nM and 500 nM, respectively.

To measure aPC formation, HEK-K293 cells stably expressing human TM on their surface cells were allowed to adhere overnight onto 24-well plates (5×10⁵ cells/well) in DMEM/F12 media containing 10% FBS (Feistritzer et al. (2006) J. Biol. Chem., 281:20077-20084). Cells were washed twice with DMEM/F12 media followed by Dulbecco PBS. Various concentrations of PF4 or protamine sulfate (PRT) were incubated with cells for 10 minutes at 37° C. followed by the addition of PC and IIa, as described above. After incubation for 45 minutes, aliquots of the reaction mixture were transferred to 96-well plates and quenched by the addition of 1 mM EDTA and 100 nM hirudin. Generation of aPC was measured after the addition of 0.5 mM 52366 and determination of the initial rate of chromogenic substrate cleavage from absorbance measurements at 405 nm on a Thermomax™ Microtiter Vmax® plate reader (Molecular Devices).

### In Vivo aPC Assay

Generation of aPC in vivo was assayed in plasma of mice as described (Kowalska et al. (2007) Blood 110:1903-1905). Mice were injected into the jugular vein. After 10 minutes, blood was drawn into sodium citrate/benzamidine (final concentration, 5 mM/50 mM, respectively), centrifuged for 10 minutes at 4500 g at 4° C., and plasma was frozen. Plasma aPC levels were subsequently measured by capture ELISA using antimouse aPC antibody and chromogenic substrate Spectrozyme® PCa (American Diagnostica) (Li et al. (2005) J. Thromb. Haemost., 3:1351-1359).

### Statistical Analysis

Differences between groups were compared using a two-tailed Student's t-test. Statistical analyses were performed using Microsoft Excel (Microsoft). Differences were considered significant when p values were ≦0.05.

### Results

Platelet factor 4 (PF4; Gene ID: 5196) increases activated protein C (aPC) generation by the thrombin (IIa)/thrombomodulin (TM) complex, both in vitro and in vivo. Potentiation of aPC generation by PF4 requires the TM-glycosaminoglycan (GAG) domain. PF4 released from platelets in mice enhances aPC generation in a model of IIa infusion and released PF4 can protect against lipopolysaccharide-induced endotoxemia.

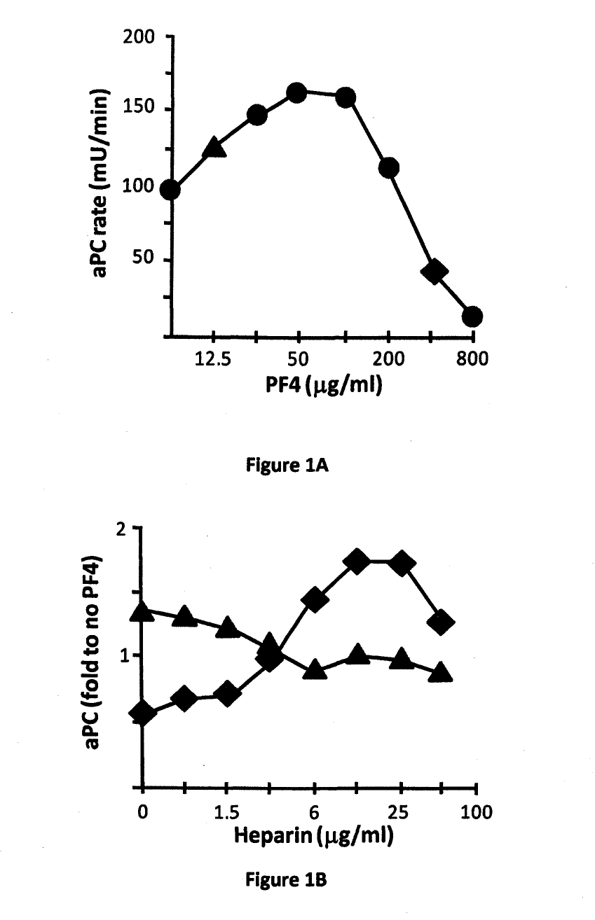

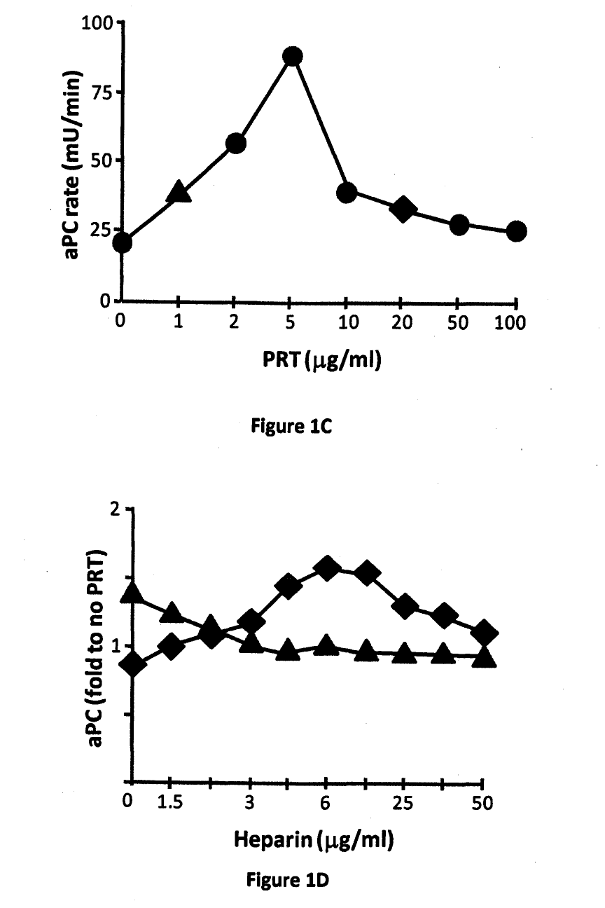

As seen in FIG. 1A, PF4 affects aPC generation following a bell-shape curve with a peak at around 50 μg/ml. Heparin has higher affinity for PF4 than other glycosaminoglycans (GAGs) and decreases effective surface PF4 concentration. At an excess of PF4, adding heparin shifts the curve back to the optimal PF4 concentration as seen with the diamonds in FIG. 1B. At low concentrations of PF4, adding any heparin simply decreases aPC generation (triangles in FIG. 1B). The same was observed for another small cationic protein, protamine sulfate (PRT). FIG. 1C shows the bell-shape curve of aPC generation as the concentration of protaimne sulfate is increased. FIG. 1D shows that at higher concentrations of PRT, a peak and then a decrease in aPC is observed. At a lower than optimal protamine sulfate concentration, adding heparin only decreases aPC generation (FIG. 1D).

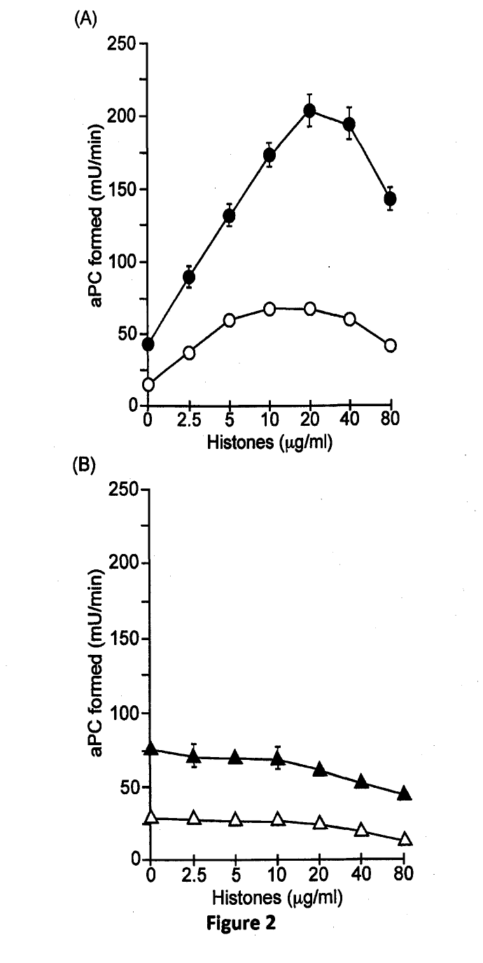

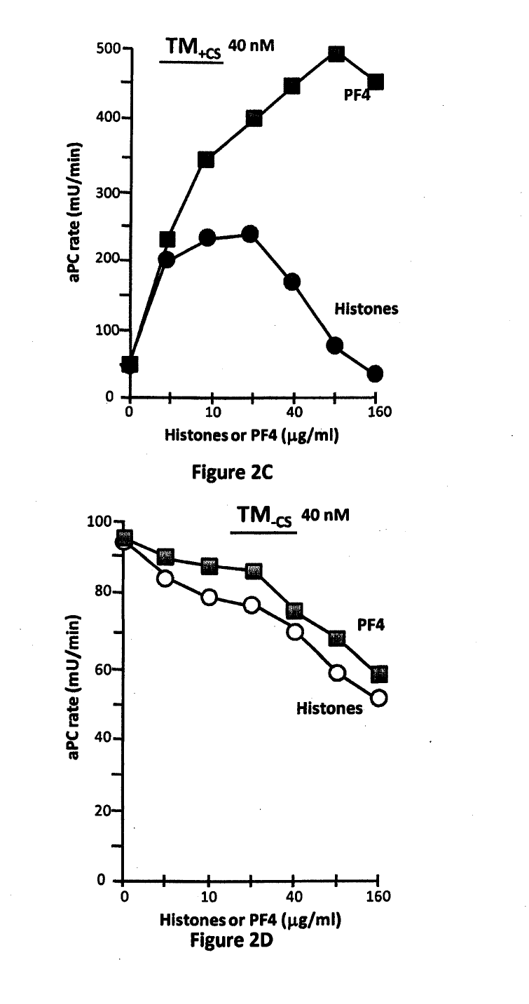

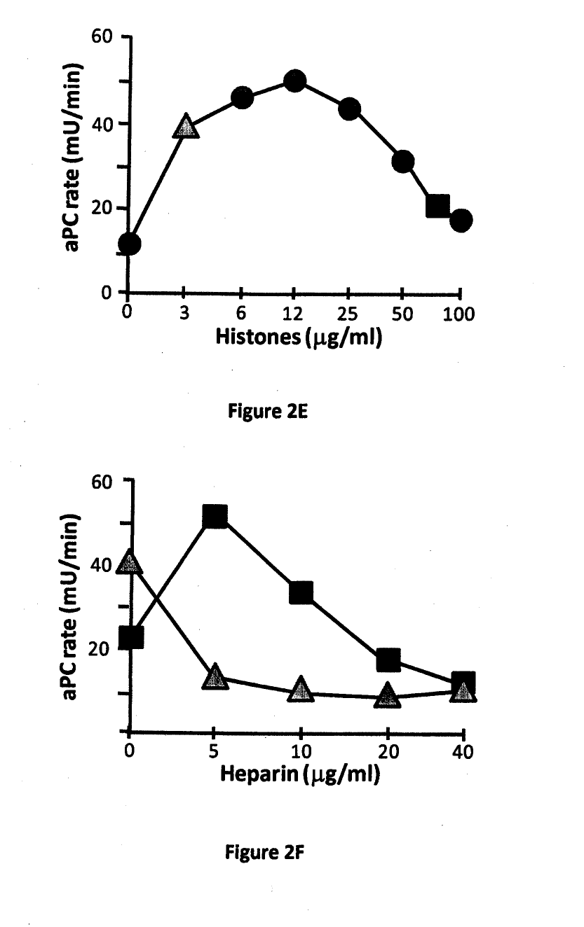

Histones are small positively charged molecules similar to PF4 and protamine sulfate. Notably, extracellular histones released in response to inflammatory challenge contribute to endothelial dysfunction, organ failure and death during sepsis (Xu et al. (2009) Nature Med., 15:1318-1321). In chromatin, DNA is wrapped around histone octamers containing a tetramer of histones H3-H4 and two dimers of histones H2A-H2B. Linker histone H1 is also highly positively charged. It is also noteworthy that DNA encompasses the positively charged histone octamer in a similar structure to that of heparin wrapped around the PF4 tetramer. To determine if histones potentiate aPC generation, the aPC assay was performed in using thrombomodulin with or without a chondroitin sulfate (CS) side chain. In the presence of the CS side chain, histones increase aPC generation in a bell-shape curve (FIG. 2A). In the absence of the CS side chain, histones had only a minor inhibitory effect (FIG. 2B). FIGS. 2C and 2D demonstrate that PF4 is more potent than histones on aPC activity, but the PF4 inhibitory effect was less pronounced in the absence of CS.

The effect of heparin on potentiation of aPC generation by mixed histones in the presence of thrombomodulin containing CS sidechains was then examined. As with other small cationic protein, when histones were present in excess, the addition of heparin shifted the curve to the left with the optimal effect at 5 μg per ml (FIGS. 2E and 2F). At below peak concentration of histones, the addition of heparin only decreased aPC generation.

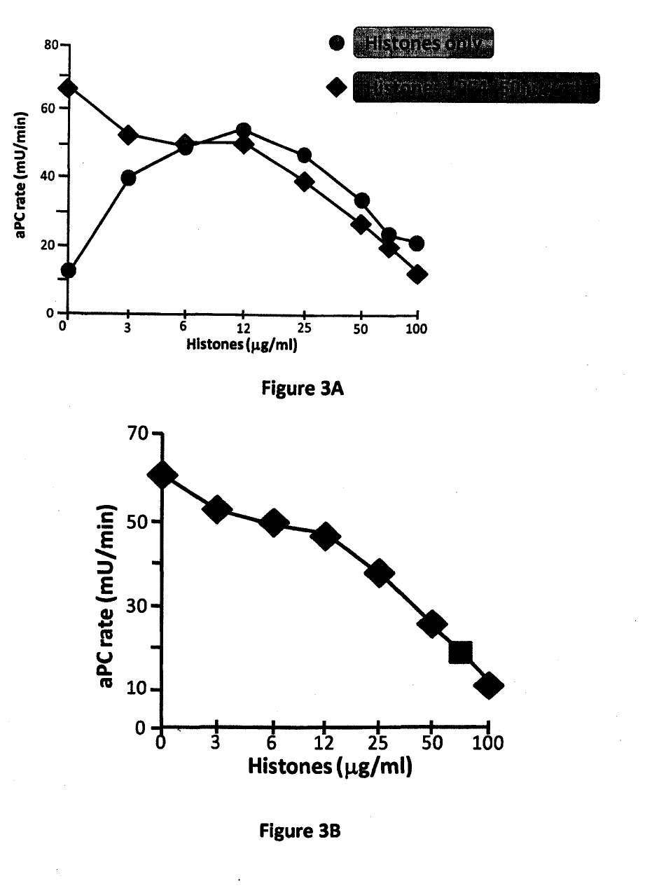

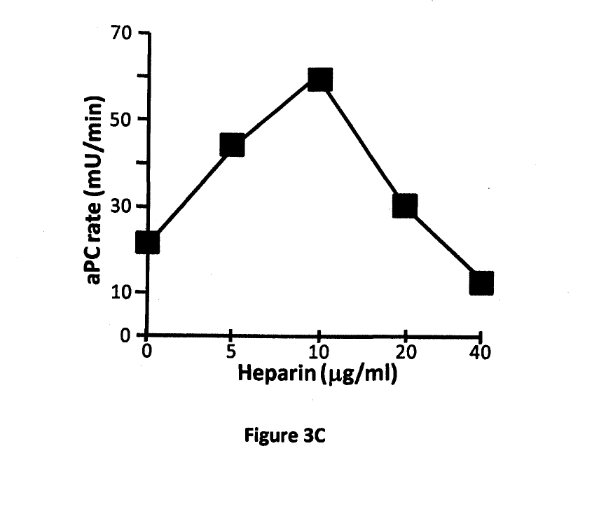

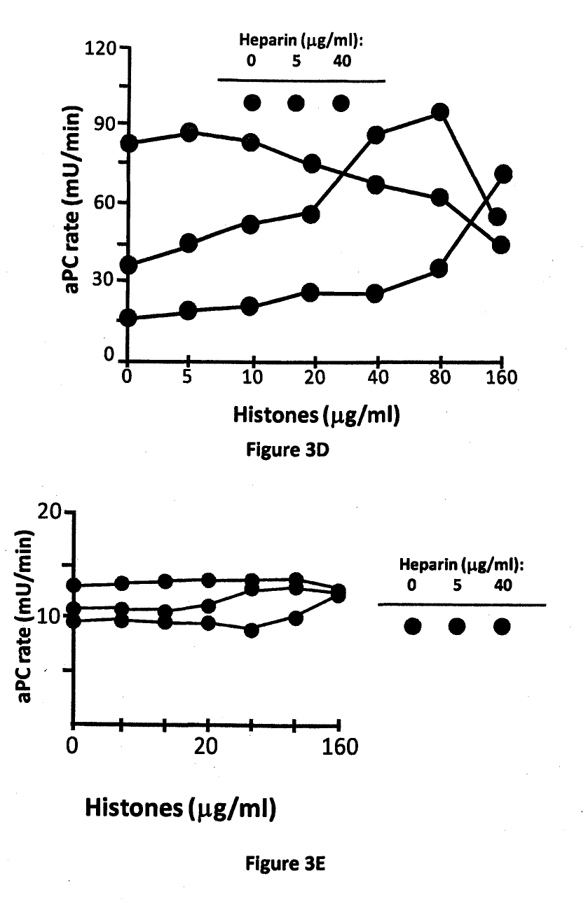

FIG. 3A demonstrates that histones and PF4 additively affect aPC generation. Adding histones with the optimal amount of PF4 present increases the total amount of positively charged small proteins and decreases aPC generation. This finding indicates that in septic patients, with high levels of circulating PF4 released, histones will only inhibit aPC generation. FIGS. 3B and 3C demonstrates that heparin affects histones/PF4 mediated aPC generation in the presence of TM_{+CS}. Under these circumstances, heparin neutralized the small cationic proteins. For example, adding heparin first enhances aPC generation before a decrease in aPC generation. More heparin is needed to optimize aPC generation in the presence of both histones and PF4 together than either alone (FIG. 3C). The effect of heparin is also seen in the presence of chondroitin sulfate-free TM, showing a similar shift to the right (FIG. 3D). However it did not have any effect in the absence of the GLA-domain of Protein C, thereby indicating that cation molecules facilitate the interaction of Protein C with thrombomodulin (FIG. 3E).

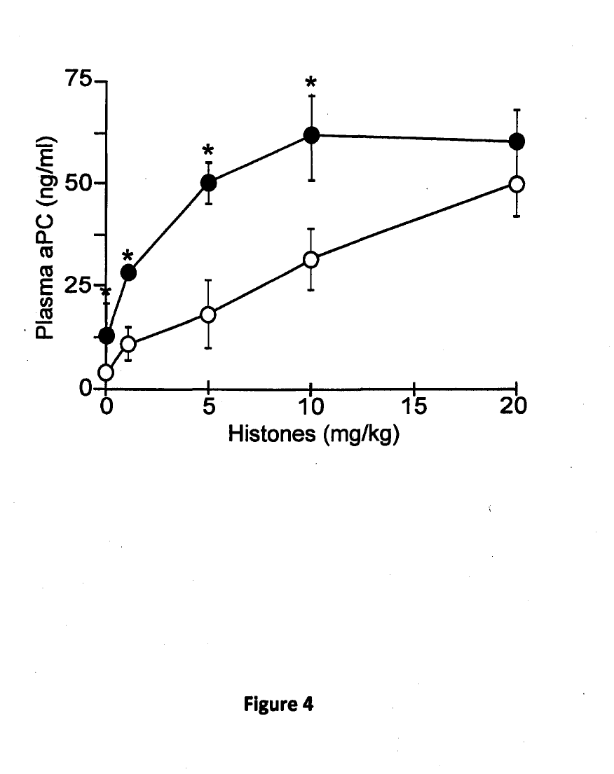

FIG. 4 demonstrates that histones plus PF4 affected aPC plasma levels compared to histones alone. Indeed, in a thrombin injection model, PF4 knockout mice were administered histones or histones+PF4 and aPC was measured. When histones are injected, plasma aPC generation is increased in a concentration dependent manner. Concomitantly, injected PF4 further increases the generation of aPC in plasma. This increase reaches a peak at a lower histone concentration.

### Example 2

Sepsis is a significant clinical challenge associated with high morbidity and mortality (Chang et al. (2010) JAMA 303:804). Key features include the development of both a coagulopathy and loss of vascular integrity. Histones, released from macrophages by endotoxin, are toxic to endothelium and that activated protein C (aPC) reduces this cytotoxic effect (Xu et al. (2009) Nat. Med., 15:1318-1321). Therapeutic interventions have included the use of heparin and also attempts to modulate the level of aPC, but with limited success (Bernard et al. (2004) Chest 125:2206-2216; Levi et al. (2007) Am. J. Respir. Crit. Care Med., 176:483-490). Increasing the level of aPC in affected patients is based on the observation that in sepsis endothelial cell is common with decreased levels of both surface-bound thrombomodulin (TM) and endothelial protein C receptor (EPCR) (Esmon, C. T. (2002) Ann. Med., 34:598-605; Told et al. (2008) Thromb. Haemost., 100:582-592). TM is an anionic molecule due to posttranslational O-linked glycanation by chondroitin sulfate (CS) (Suzuki et al. (1987) EMBO J., 6:1891-1897). The generation of aPC by the thrombin (IIa)/TM complex and presence of EPCR is crucial for limiting thrombosis and stabilizing vascular integrity (Esmon, C. T. (1989) J. Biol. Chem., 264:4743-4746; Esmon, C. (2000) Crit. Care Med., 28:S44-48; Weiler et al. (2008) Curr. Opin. Hematol., 15:487-493).

Generation of aPC by IIa complexed to TM that contains CS sidechains (TM_{CS}) can be accelerated, both in vitro and in vivo, by positively-charged molecules like platelet factor 4 (PF4) or protamine sulfate (PRT) (Slungaard et al. (1994) J. Biol. Chem., 269:25549-25556; Slungaard et al. (2003) Blood 102:146-151). Binding studies using surface plasmon resonance (Dudek et al. (1997) J. Biol. Chem., 272:31785-31792) have confirmed a strong interaction of PF4 with both TM's glycosaminoglycan (GAG) moiety and PC's G1a domain. In different murine transgenic lines, platelet PF4 content is proportional to enhanced aPC generation after IIa infusion and PF4 released from platelets is protective against lipopolysaccharide (LPS)-induced endotoxemia (Kowalska et al. (2007) Blood 110:1903-1905). Importantly, PF4 affects aPC generation by IIa-TM_{CS} along a bell-shaped curve with an optimal specific PF4:TM_{CS} molar ratio. Unfractionated heparin (UFH), which binds PF4 with greater affinity than does CS, reduces the effective PF4:TM_{CS} molar ratio, effectively shifting the aPC-generation bell-shaped curve to the right. Thus added UFH enhances aPC formation at high PF4 concentrations, while inhibiting at low PF4 concentrations (Kowalska et al. (2011) Blood 118:2882-2888). Similar effect of UFH was seen for PRT modulation of aPC.

Histones have been shown to be released from dying cells and worsen the outcome in sepsis in humans (Zeerleder et al. (2003) Crit. Care Med., 31:1947-1951). Injections of aPC (Xu et al. (2009) Nat. Med., 15:1318-1321) or UFH (Fuchs et al. (2011) Blood 118:3708-3714) reverse histone-induced lethality in mice. Relevant to these studies, histones have been shown to increase plasma IIa generation by impairing TM-dependent PC activation (Ammollo et a. (2011) J. Thromb. Haemost., 9:1795-1803). Since histones are positively-charged small proteins like PF4 and PRT, it was expected that they would actually enhance aPC generation by IIa complexed to TM_{CS} at low levels and only inhibit aPC generation at high levels. The interactions between histones and TM_{CS} were examined with and without added PF4. It was reasoned that in severe sepsis with histones released, large amounts of PF4 may also be present from activated platelets, including by histones, which are known to activate platelets (Fuchs et al. (2011) Blood 118:3708-3714). Moreover, in severe sepsis, heparin would likely be used in patients and this could also influence the effects of PF4 and histones on aPC generation.

Here, it is shown that histones moderately impair aPC generation in the presence of TM that lacks CS sidechains (TM_{-CS}). However, in the presence of TMcs, histones affect aPC generation similarly to PF4 following a bell-shape curve of enhanced aPC generation with a peak enhancement of ˜5-fold. The effect of PF4 and histones released is additive on aPC generation both in vitro and in vivo in mice. The effective concentration of both is reduced by the co-presence of UFH, but this effect was also associated with a significant anticoagulant activity in vivo. Therefore, partially-desulfated, 2-O,3-O desulfated heparin (ODSH) that has been noted to bind well to PF4 (Joglekar et al. (2012) Thrombosis Haemostasis 107:717-725) yet has ˜60-fold less antithrombin activity (Fryer et al. (1997) J. Pharm. Exp. Ther., 282:208-219), was tested to see its efficacy at binding to histones and affecting aPC generation in its presence. It was found that ODSH had a similar effect as UFH on histone/aPC generation in vitro and in vivo, but without the anticoagulant effect and allowing for the correction of aPC generation in a lethal histone infusion murine model.

Histones are known to be detrimental in late sepsis, and both activated protein C (aPC) and heparin reverse this effect. It is shown that histones modulate aPC formation in vitro and in vivo in a manner similar to other positively-charged molecules, particularly, platelet factor 4 (PF4). At low doses, histones actually enhances aPC generation up to several-fold. In vitro, histones effect on aPC formation is additive with PF4, and this effect is further modulated by the heparinoids, unfractionated heparin (UFH) and oxygen-desulfated UFH (ODSH) with its marked decrease in anticoagulant activity. These conclusions were supported by aPC generation studies in mice infused with histones, PF4 and heparinoids. Importantly, while UFH and ODSH can both reverse the lethality of high-dose histone, only mice treated with ODSH demonstrated corrected activated partial thromboplastin time (aPTT) and significant levels of formed aPC. The instant data provide a model of how histones affect aPC generation, and when and how heparinoids, especially ODSH, are beneficial. These studies provide new insights into the complex interactions controlling aPC generation in sepsis, and the benefit of monitoring both aPC and aPTT levels concurrent with ODSH therapy to improve outcome of this life-threatening disorder.

### Materials and Methods

### Reagents

All reagents, including high molecular-weight, porcine UFH, sodium salt (specific activity 196 U/mg), unless specified otherwise, were from Sigma-Aldrich (St. Louis, Mo.). Heparinoid ODSH (2-O, 3-O desulfated heparin) was a obtained from ParinGenix Inc. ODSH is also described, along with methods of synthesis, in Rao et al. (am. J. Cell Physiol. (2010) 299:C97-C110) and Fryer et al. (J. Pharmacol. Exp. Ther. (1997) 282:208-219). Chromogenic substrate S2366 was obtained from Chromogenix/diaPharma (West Chester, Ohio), recombinant hirudin was obtained from Calbiochem (Billerica, Mass.), and protamine sulfate (PRT) was obtained from American Pharmaceutical Partners (Schaumburg, Ill.). Mixed histones for in vitro studies were obtained from Roche Diagnostic and those used for studies in vivo were obtained from Sigma-Aldrich. Histones H1 and H4 were obtained from New England Biolabs (Ipswich, Mass.). Human soluble TM was purified from TM-expressing HEK293 cells by anion-exchange chromatography and affinity chromatography on IIa-sepharose (Lu et al. (2005) J. Biol. Chem., 280:15471-15478). Non-glycosylated, low molecular weight TM was separated from glycosylated, high molecular weight TM by high performance anion exchange chromatography to yield TM_{-CS} (Parkinson et al. (1990) J. Biol. Chem., 265:12602-12610; Lu et al. (2005) J. Biol. Chem., 280:15471-15478). The different forms of TM were validated by Western blot (Kowalska et al. (2011) Blood 118:2882-2888). Rabbit TM was purchased from Hematologic Technologies (Essex Junction, Vt.). Human protein C, was isolated from plasma (Baugh et al. (1996) J. Biol. Chem., 271:16126-16134), and further purified by immunoaffinity chromatography using the Ca²⁺-dependent monoclonal antibody HPC4 (Stearns et al. (1988) J. Biol. Chem., 263:826-832).

### In Vitro aPC Assay

Generation of aPC was assayed in 96-well plates generally as described above. Briefly, rabbit or human sTM_{CS} or sTM_{-CS}, was mixed with various amounts of histones, PF4 and/or heparinoid for 10 minutes in assay buffer (final concentration, 20 mM Tris, 100 mM NaCl, 1 mM CaCl₂, 0.1% BSA, pH 7.5). Final concentrations of TMs were 0.5-40 nM. PC (final concentration, 500 nM) was added for an additional 10 minutes followed by addition of IIa at a final concentration of 0.2 or 2 nM and then incubated for 15 minutes. All incubations were done at 37° C. The reaction was quenched with 1 mM EDTA and 100 nM hirudin. Concentrations of aPC formed in the quenched samples were inferred from initial rate measurements after the addition of 0.5 mM S2366. Initial rates of chromogenic substrate cleavage were determined by measuring absorbance at 405 nm using a ThermoMax™ Microtiter Vmax® plate reader (Molecular Devices; Sunnyvale, Calif.).

### In Vivo aPC Measurements

PF4 knockout (PF4^{KO}) mice were previously generated and characterized (Eslin et al. (2004) Blood 104:3173-3180). WT mice were also on C57B16 background. All mice had been backcrossed>10 times onto a C57B16 background. Mice studied were 8-12 weeks of age. All animal experiments were approved by the Children's Hospital of Philadelphia's institutional animal care and use committee.

In vivo aPC generation was assayed in plasma of mice generally as described above. Specifically, mice were injected via the jugular vein over 2 minutes with murine IIa at 8 U/kg (Kowalska et al. (2007) Blood 110:1903-1905) concurrently with histones (0-20 mg/kg) in the absence or presence of 5 mg/kg of PF4. After 10 minutes, blood was drawn into sodium citrate/benzamidine (final concentration 5 mM/50 mM, respectively), centrifuged for 10 minutes at 5,000 rpm at 4° C. and plasma frozen. In some experiments, histones at 60 mg/kg, was injected in the absence or presence of 5 mg/kg of PF4 and blood was drawn after 60 minutes. Some animals were pre-injected with UFH or ODSH 10 minutes before injection of histones. Plasma aPC levels were measured by capture ELISA (Li et al. (2005) J. Thromb. Haemost., 3:1351-1359) using an anti-mouse aPC antibody and chromogenic substrate Spectrozyme® PCa (American Diagnostica; Stamford, Conn.).

### Statistical Analysis

Statistical analyses were performed as above.

### Results

### Modulation of aPC Formation by Histones

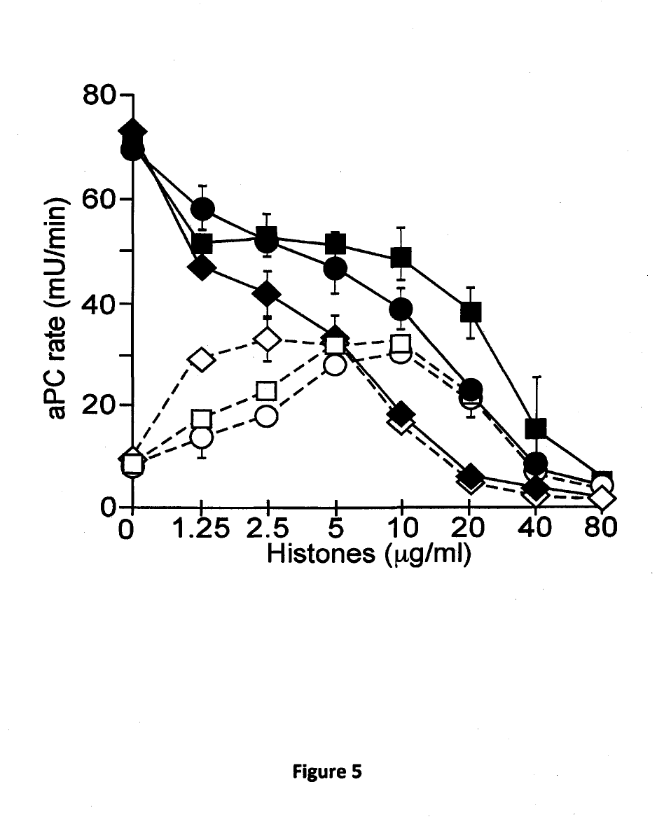

Positively-charged small molecules enhance aPC generation with a bell-shaped response in the presence of sTM_{CS}. Herein, it was tested whether histones, which are also small, positively-charged proteins, would have a similar effect. A histone mixture of all four histones was used to duplicate the forms likely released in vivo during sepsis (Zeerleder et al. (2003) Crit. Care Med., 31:1947-1951). It is shown that aPC generation increases upon addition of increasing amount of histones, peaking at 10-20 μg/ml of added histones (FIG. 2A), in the presence of either low (0.5 nM) or high (40 nM) concentrations of sTM_{CS}. Peak rates of aPC formation were ˜5-fold greater than that seen in the absence of histones. In contrast, enhanced aPC formation was not seen with TM_{-CS} (FIG. 2B). Instead, histones had only a minor inhibitory effect, consistent with other reports (Ammollo et al. (2011) J. Thromb. Haemost., 9:1795-1803). Experiments using human recombinant individual histones H1 and H4 gave similar results (FIG. 5). Small differences in peak activity of various histones were noted and could be attributed to the difference in molecular weight, ability of individual histones to form higher molecular weight complexes with sTM_{CS} or the small differences in amino acid composition and charge.

### PF4 and Heparin Influence Histone-Mediated aPC Generation

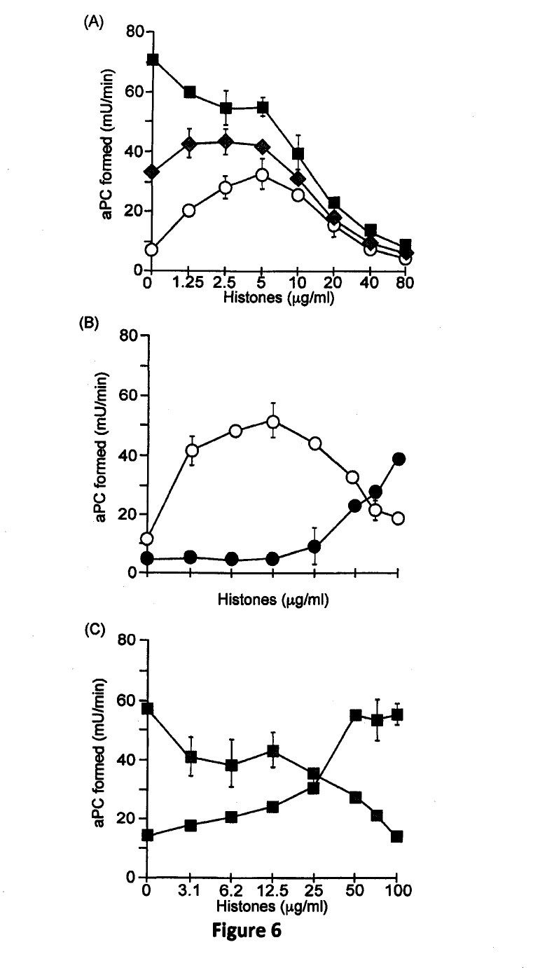

Since in sepsis both histones and LPS activate platelets that then release their α-granules content, including PF4 (Fuchs et al. (2011) Blood 118:3708-3714; Kowalska et al. (2007) Blood 110:1903-1905), the possibility that PF4 and histones act additively to regulate PC activation was assessed. In the absence of histones (FIG. 6A, 0 histones), inclusion of PF4 at below-peak PF4 concentration (3 μg/ml) increased aPC generation>3-fold (FIG. 6A, gray diamonds), but in the presence of a higher (25 μg/ml), near-peak concentration of PF4 (FIG. 6A, black squares), increase was around 7-fold (Kowalska et a. (2011) Blood 118:2882-2888). Histones increased aPC generation with a peak at ˜2.5 μg/ml in the presence of 3 μg/ml of PF4 (FIG. 6A, grey diamonds), compared to the peak of ˜5 μg/ml in the absence of PF4, (FIG. 6A, open circles). In the presence of near-peak concentration of PF4 (FIG. 6A, black squares) further addition of histones only inhibited aPC generation at all concentrations of histones studied. Individual histones were also similarly affected by PF4 (FIG. 5). In the presence of UFH (5 μg/ml), peak aPC generation from histone plus PF4 was shifted to the right (FIG. 6C) just as seen with histones or PF4 alone (FIG. 6B; Kowalska et al. (2011) Blood 118:2882-2888).

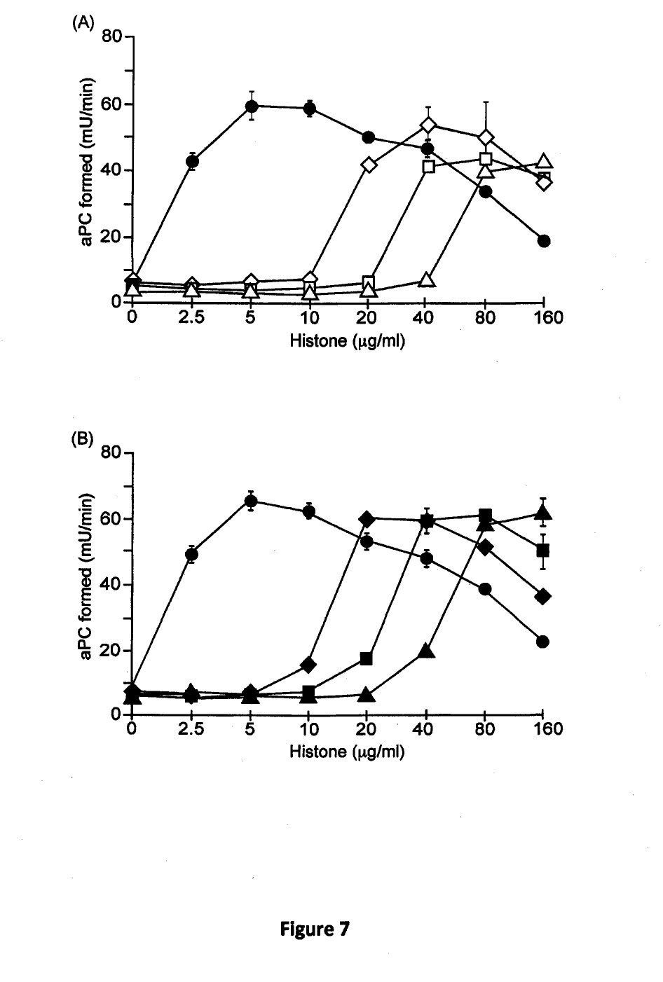

The effect of various concentrations of UFH or ODSH on aPC generation was also studied in the presence of various concentrations of histones (FIGS. 7A and 7B, respectively). Levels of released histones may vary from no to low histones in less severe sepsis to high concentrations in severe sepsis. As shown on FIG. 7, at low levels of histones (0-10 μg/ml) all doses of heparin or ODSH tested inhibited aPC generation. At higher (>10 μg/ml) levels of histones, in the absence of heparinoids, results in decreased aPC formation (FIGS. 7A and 7B, grey circles), the presence of UFH or ODSH restored high levels of aPC generation. Higher concentrations of heparinoids were needed to restore peak of aPC formation as concentration of histones increased. In conditions simulating late sepsis, when high doses of PF4 and histones are present, both heparin and ODSH enhanced aPC generation and only suppressed aPC generation at high doses (FIG. 7B, closed squares).

### In Vivo Effects of Histones and PF4 on aPC Generation in Mice

To establish the physiological correlates for the observations in vivo, the ability of intravenously injected histones and PF4 along with low dose IIa to modulate aPC formation was examined in mice (Kowalska et al. (2007) Blood 110:1903-1905). PF4^{KO} mice were used in these studies to eliminate the confounding influence of variable platelet-released PF4 by the injected histones as histones activate platelets (Fuchs et al. (2011) Blood 118:3708-3714). At low levels of 0-20 mg/kg, histones co-injected with IIa increased plasma aPC generation in a concentration-dependent manner (FIG. 4). Concurrent injection of PF4 with the IIa and low doses of histones significantly lead to higher aPC levels at these low histone levels.

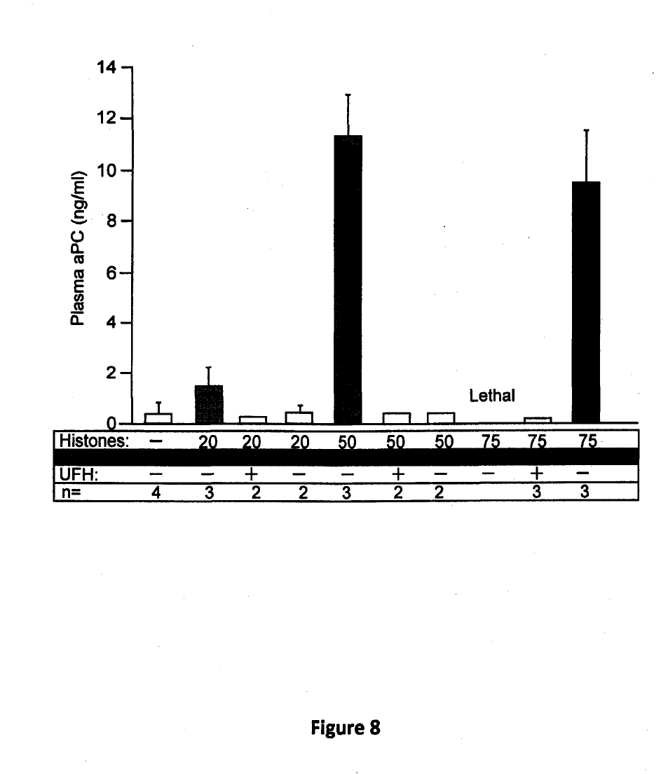

At a higher dose of histones (50 mg/kg), co-injection of histones with IIa was lethal in less than 60 minutes in PF4^{KO} mice, similar to that reported of injecting 75 mg/kg of histone in WT mice (Xu et al. (2009) Nat. Med., 15:1318-1321; Fuchs et al. (2011) Blood 118:3708-3714). Therefore, the effects of 50 mg/kg of histones injected without IIa in WT mice were studied (FIG. 8). This histone dose increased the level of circulating aPC as compared to control buffer or 20 mg/kg of histone (FIG. 8, grey bars), and added UFH or ODSH suppressed detectable aPC generation. At a higher dose of 75 mg/kg of histones alone, all of the treated WT mice died similar to that reported (Fuchs et al. (2011) Blood 118:3708-3714). Co-injection of UFH at 50 mg/kg has been reported to prevent these deaths, and a similar outcome was found after a similar dose of UFH or ODSH. However, in contrast to UFH (FIG. 8, white bars), aPC levels were raised in mice pretreated with ODSH (dashed bars).

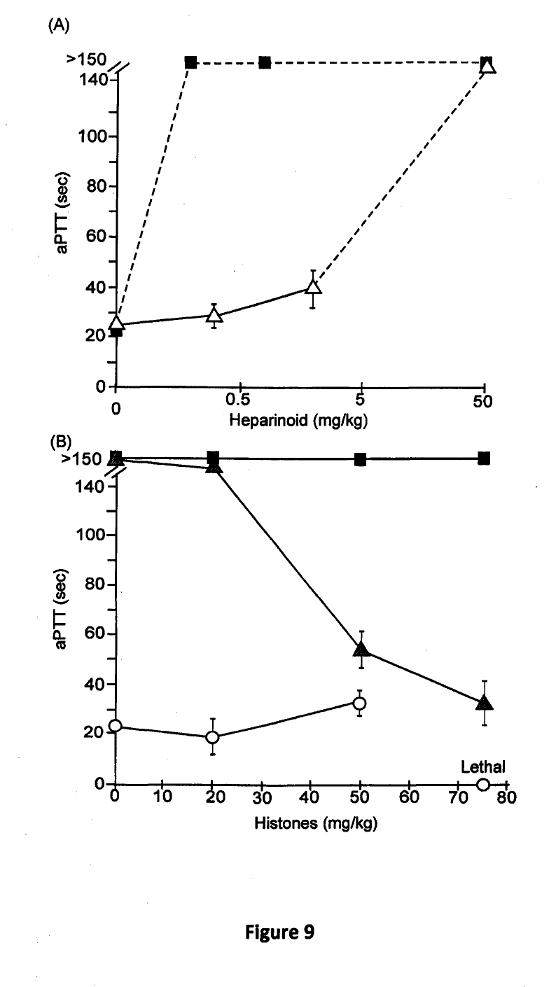

The dose of UFH needed to block the lethality of 75 mg/kg of histones in mice is ˜100-fold greater than that needed to clinically heparinize individuals. Consistent with this information, UFH at concentrations of 10 and 100-times lower than needed to reverse histone lethality result in marked prolongation of aPTT in mice not exposed to histones (FIG. 9A, black squares). In contrast, in non-histone treated mice, the dose of ODSH required to markedly prolong the aPTT was the same as needed to reverse histone lethality (FIG. 9A, open triangles). aPTT was also measured in mice injected with 50 mg/kg of either UFH or ODSH followed 10 minutes later by injection of increasing concentrations of histones. It was found that in the presence of high (sub-lethal and lethal) concentrations of histones, severe prolongation of aPTT by the ODSH, but not by the UFH, was reversed (FIG. 9B) consistent with sufficient neutralization of the ODSH by available histories and released platelet PF4 to eliminate ODSH's anticoagulant affect but not heparin's.

Septic patients have activation of blood coagulation, consumption of anticoagulant factors and increased platelet activation (Muller-Berghaus et al. (1999) Thromb. Haemost., 82:706-712; Toh et al. (2003) Hematology 8:65-71). In the late state of sepsis, endothelial cell dysfunction is a key element, with decreased TM and EPCR levels on the surface of endothelial cells (Esmon, C. T. (2002) Ann. Med., 34:598-605; Told et al. (2008) Thromb. Haemost., 100:582-592) and impaired aPC generation. Recombinant human sTM (ART-123) has been used for the treatment of DIC associated with hematologic malignancy or infection (Saito et al. (2007) J. Thromb. Haemost., 5:31-41) and with sepsis (Nagato et al. (2009) Crit. Care Med., 37:2181-2186). TM exists as TM_{CS} or lacking this GAG moiety, TM_{-CS}. In various vascular beds both of those forms may be expressed to different extents in different species (Lin et al. (1994) J. Biol. Chem., 269:25021-25030). It has been shown that the TM_{CS} form has a higher affinity for IIa than TM_{-CS} and is more effective in inhibition of clotting (Parkinson et al. (1990) J. Biol. Chem., 265:12602-12610). Mice studies have shown that aPC's efficacy is linked to the ability of aPC to activate cytoprotective effects by signaling through EPCR and protease activated receptor-1 (Weiler et al. (2008) Curr. Opin. Hematol., 15:487-493). Thus, locally increased levels of aPC generation can be more beneficial in severe sepsis than treatment with systemic infusion of PC or aPC.

aPC generation from TM_{CS} can be upregulated several fold by several different positively-charged small proteins, specifically PF4 released from activated platelets and iatrogenic infused PRT, and that this upregulation follows a bell-shaped curve with regard to the positive small molecule's concentration and can be neutralized by large negatively-charged molecules like heparin (Kowalska et al. (2007) Blood 110:1903-1905). Herein, it is shown that histones, which are released in large amounts as sepsis progress, behave in a similar manner as PF4. Like other small, highly positive small proteins, histones interact with the GAG-sidechain of TM and enhance IIa/TM aPC generation activity multiple-fold. This positive-charge effect follows a bell-shaped curve and likely peaks at a 1:1 molar ratio of histones and TM_{CS}. Thus, unlike prior studies in mice, the instant data provides a more detailed study of histones' affects on at least aPC generation as well as the benefit of its partial neutralization in sepsis.

In mild sepsis, PF4 is released from activated platelets. Depending on the platelet count and PF4 content, both of which can vary significantly (Lambert et al. (2007) Blood 110:1153-1160), aPC generation would occur on intact endothelial cell TM_{CS}. At the same time, histone levels are low or absent. Infused UFH or ODSH in this setting is likely to diminish aPC generation and be of no therapeutic benefit. During moderate sepsis, more platelets may be activated, and the end result is that PF4 levels may be higher. On top of this neutrophil release of chromatin and formation of neutrophil enhancement traps (NETS) may begin to form (Fuchs et al. (2010) Proc. Natl. Acad. Sci., 107:15880-15885) and histones released. The levels of PF4 and histone may lead to near-optimal aPC formation by IIa/TM_{CS}. In this case, UFH or ODSH doses may either increase or decrease aPC generation depending on the aggregate dose of positively-charged small proteins are present. In severe sepsis, large amounts of histones are circulating as well as free PF4 and the result is suppressed aPC generation. In this setting, added UFH or ODSH may have negligible affects on aPC generation because of inadequate dosing or may improve aPC levels if properly titered. The results shown in moderate and severe sepsis along with the data shown in FIGS. 8 and 9 may explain why heparin has been reported to be of variable utility in sepsis. The instant data supports measuring both aPTT and aPC levels in sepsis to titer the effects of heparinoids on outcome. They also demonstrate that ODSH with its lower anticoagulants affects offer the possibility of titering its level to achieve a significant aPC level while avoiding bleeding complications.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for treating a coagulopathy in a subject in need thereof, said method comprising:
a) determining the concentration of platelet factor 4 (PF4) and, optionally, histones in a biological sample obtained from said subject, and
b) administering to the subject a therapeutically effective amount of at least one negatively charged compound or protein when the concentration of PF4 and, optionally, histones determined in step a) is greater than the optimal concentration for producing activated protein C (aPC) or administering to the subject a therapeutically effective amount of at least one positively charged compound or protein when the concentration of PF4 and, optionally, histones determined in step a) is lower than the optimal concentration for producing aPC.

2. The method of claim 1, comprising determining the concentration of platelet factor 4 (PF4) and histones in said biological sample.

3. The method of claim 1, wherein said negatively charged compound or protein is heparin or heparinoid.

4. The method of claim 3, wherein said heparin is oxygen desulfated heparin.

5. The method of claim 1, wherein said positively charged compound or protein is protamine sulfate (PRT), PF4, or histones.

6. The method of claim 1, wherein said method further comprises administering thrombomodulin with chondroitin sulfate side chains.

7. The method of claim 1, wherein the biological sample is blood or a fraction thereof.

8. The method of claim 1, further comprising determining the concentration of heparin.

9. The method of claim 8, wherein the concentration of heparin is determined by activated partial thromboplastin time (aPTT).

10. The method of claim 1, further comprising determining the concentration of endothelial protein C receptor (EPCR).

11. The method of claim 1, further comprising determining the concentration of aPC.

12. The method of claim 1, wherein said coagulopathy is disseminated intravascular coagulopathy (DIC).

13. The method of claim 12, wherein said DIC is associated with sepsis or acute respiratory distress syndrome (ARDS).

14. The method of claim 1, wherein said method further comprises the administration of at least one other anti-sepsis agent.

15. The method of claim 14, wherein said anti-sepsis agent is selected from the group consisting of activated protein C, anti-thrombin III, bactericidal/permeability-increasing protein (BPI), anti-infectives, and antibiotics.

16. The method of claim 15, wherein said antibiotic is selected from the group consisting of β-lactam, penicillin, cephalosporin, vancomycin, bacitracin, macrolide, erythromycin, lincosamide, clindomycin, chloramphenicol, tetracycline, immunocycline, chlortetracycline, oxytetracycline, demeclocycline, methacycline, doxycycline, minocycline, aminoglycoside, gentamicin, amikacin, neomycin, amphotericin, cefazolin, clindamycin, mupirocin, sulfonamide, trimethoprim, rifampicin, metronidazole, quinolone, novobiocin, polymixin, gramicidin, imipenem, meropenem, cefoperazone, cefepime, nafcillin, linezolid, aztreonam, piperacillin, tazobactam, ampicillin, sulbactam, clindamycin, metronidazole, levofloxacin, carbapenem, linezolid, rifampin, and metronidazole.

17. The method of claim 1, wherein said administration is intravenously.

18. The method of claim 1, further comprising determining the optimal concentration of PF4 for producing aPC in said biological sample.
